(19) United States
(12) Patent Application Publication
(10) Pub. No.: US 2017/0335305 A1
(43) Pub. Date: Nov. 23, 2017

(54) GENUS GLUCONACETOBACTER MICROORGANISM HAVING ENHANCED CELLULOSE PRODUCTIVITY, METHOD OF PRODUCING CELLULOSE USING THE SAME, AND METHOD OF PRODUCING MICROORGANISM

(71) Applicant: Samsung Electronics Co., Ltd., Suwon-si (KR)

(72) Inventors: Jinsuk Lee, Seoul (KR); Hongsoon Rhee, Suwon-si (KR); Jinkyu Kang, Hwaseong-si (KR); Jinhwan Park, Suwon-si (KR)

(21) Appl. No.: 15/413,599

(22) Filed: Jan. 24, 2017

(30) Foreign Application Priority Data
May 17, 2016 (KR) 10-2016-0060209

(51) Int. Cl. C12N 9/52; C12P 19/12; C12P 19/18; C12N 9/04

(52) U.S. Cl. CPC C12N 9/52; C12Y 304/25002; C12Y 101/01047; C12N 9/0006; C12P 19/12; C12P 19/18

(57) ABSTRACT

Provided are a genus Gluconacetobacter microorganism having enhanced cellulose productivity, a method of producing cellulose using the same, and a method of producing the microorganism.

### CROSS-REFERENCE TO RELATED APPLICATION

This application claims the benefit of Korean Patent Application No. 10-2016-0060209, filed on May 17, 2016, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 21,265 Byte ASCII (Text) file named “726809_ST25.TXT,” created on Jan. 24, 2017.

### BACKGROUND

### 1. Field

The present disclosure relates to a genus Gluconacetobacter microorganism having enhanced cellulose productivity, a method of producing cellulose using the same, and a method of producing the microorganism.

### 2. Description of the Related Art

In cellulose produced by culturing a microorganism, glucose exists as a primary structure, β-1,4 glucan, which forms a network structure of fibril bundles. This cellulose is also called ‘bio-cellulose or microbial cellulose’.

Unlike plant cellulose, microbial cellulose is pure cellulose entirely free of lignin or hemicellulose. Microbial cellulose is 100 nm or less in width and has high water absorption and retention capacity, high strength, high elasticity, and high heat resistance compared to plant cellulose. Due to these characteristics, microbial cellulose is useful in a variety of fields, such as cosmetics, medical products, dietary fibers, audio speaker diaphragms, and functional films.

Therefore, to meet the demands for microbial cellulose, there is a need to produce microorganisms having enhanced cellulose productivity. This invention provides such a microorganism.

### SUMMARY

An aspect of the invention provides a Gluconacetobacter microorganism comprising a genetic modification that increases the activity of ATP-dependent protease ATPase (HsIUV) subunit HsIU.

Another aspect of the invention provides a method of producing cellulose using a Gluconacetobacter microorganism comprising a genetic modification that increases the activity of ATP-dependent protease ATPase (HsIUV) subunit HsIU.

Still another aspect of the invention provides a method of producing a Gluconacetobacter microorganism comprising a genetic modification that increases the activity of ATP-dependent protease ATPase (HsIUV) subunit HsIU.

### BRIEF DESCRIPTION OF THE DRAWINGS

These and/or other aspects will become apparent and more readily appreciated from the following description of the embodiments, taken in conjunction with the accompanying drawings in which:

FIG. 1 shows the amount of cellulose nanofiber (CNF) produced by G. xylinus and G. xylinus (HsIU) strains under static culture; and

FIG. 2 show the amount of cellulose nanofiber (CNF) produced by G. xylinus (Δgdh) and G. xylinus (Δgdh-HsIU) strains under static culture.

### DETAILED DESCRIPTION

The term “increase in activity” or “increased activity” or similar terms, as used herein, may refer to a detectable increase in an activity of a cell, a protein, or an enzyme. The “increase in activity” or “increased activity” or the like may also refer to an activity level of a modified (e.g., genetically engineered) cell, protein, or enzyme that is higher than that of a comparative cell, protein, or enzyme of the same type, such as a cell, protein, or enzyme that does not have a given genetic modification (e.g., original or “wild-type” cell, protein, or enzyme). The phrase “activity of a cell” may refer to an activity of a particular protein or enzyme of a cell. For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more than an activity of a non-engineered cell, protein, or enzyme of the same type, i.e., a wild-type or “parent” cell, protein, or enzyme. A cell having an increased activity of a protein or an enzyme may be identified by using any method known in the art.

An increase in activity of an enzyme or a polypeptide may be achieved by an increase in the expression or specific activity thereof. The increase in the expression may be achieved by introduction of a polynucleotide encoding the enzyme or the polypeptide into a cell, by an increase in a copy number, or by a mutation in the regulatory region of the polynucleotide. The microorganism to be introduced with the gene may include the gene autonomously or may not include the gene. The polynucleotide encoding the enzyme may be operably linked to a regulatory sequence that allows expression thereof, for example, a promoter, an enhancer, a polyadenylation region, or a combination thereof. The polynucleotide whose copy number is increased may be endogenous or exogenous. The endogenous gene refers to a gene which is included in a microorganism prior to introducing the genetic modification (e.g., a native gene). The exogenous gene refers to a gene that is introduced into a cell from the outside. The introduced gene may be homologous or heterologous with respect to the host cell. The term “heterologous” means that the gene is foreign or “not native” to the species.

The “increase in the copy number” of a gene may be caused by introduction of an gene or amplification of a gene already existing in a microorganism, and may be achieved by genetically engineering a cell so that the cell is allowed to have a gene (e.g., extra copy of a gene) that does not exist in a non-engineered cell. The introduction of the gene may be mediated by a vehicle such as a vector. The introduction may be a transient introduction in which the gene is not integrated into a genome, or an introduction that results in integration of the gene into the genome. The introduction may be performed, for example, by introducing a vector into the cell, in which the vector includes a polynucleotide encoding a target polypeptide, and then replicating the vector in the cell, or by integrating the polynucleotide into the genome.

The introduction of the gene may be performed by a known method, such as transformation, transfection, and electroporation. The gene may be introduced via a vehicle or in itself. As used herein, the term “vehicle” or “vector” refers to a nucleic acid molecule that is able to deliver other nucleic acids linked thereto into a cell. As a nucleic acid sequence mediating introduction of a specific gene, the vehicle used may be a vector, a nucleic acid construct, and a cassette. Examples of the vector may include a plasmid (e.g., plasmid expression vector), and a virus (e.g., viral expression vector), such as a replication-defective retrovirus, adenovirus, adeno-associated virus, or a combination thereof.

As used herein, the gene manipulation may be performed by any molecular biological methods known in the art.

The term “inactivated” or “decreased” activity, as used herein, means that a cell has an activity of an enzyme or a polypeptide that is lower than the same activity measured in a parent cell (e.g., a non-genetically engineered cell). Also, “inactivated” or “decreased” activity means that an isolated enzyme or a polypeptide has an activity that is lower than the same activity of an original or a wild-type enzyme or polypeptide. For example, a modified (e.g., genetically engineered) cell or enzyme has enzymatic activity of converting a substrate to a product, which shows about 20% or more, about 30% or more, about 40% or more, about 50% or more, about 55% or more, about 60% or more, about 70% or more, about 75% or more, about 80% or more, about 85% or more, about 90% or more, about 95% or more, or about 100% decrease, compared to that of a cell or enzyme that does not have the modification, i.e., a parent cell or a “wild-type” cell or enzyme. Decreased activity of an enzyme or a cell may be confirmed by any method known in the art. The inactivation or decrease includes situations in which the enzyme has no activity, the enzyme has decreased activity even though the enzyme is expressed, or the enzyme-encoding gene is not expressed or expressed at a low level compared to a cell having a non-modified gene, i.e., a parent cell or a wild-type cell.

The term “parent cell” refers to an original cell, for example, a non-genetically engineered cell of the same type with respect to an engineered microorganism. With respect to a particular genetic modification, the “parent cell” may be a cell that lacks the particular genetic modification, but is identical in all other respects. Thus, the parent cell may be a cell used as starting material to produce a genetically engineered microorganism having an inactivated or decreased activity of a given protein (e.g., a protein having a sequence identity of about 95% or more to the GDH protein) or a genetically engineered microorganism having an increased activity of a given protein (e.g., a protein having a sequence identity of about 95% or more to the HsIU protein). By way of further illustration, with respect to a cell in which a gene encoding GDH has been modified to reduce GDH activity, the parent cell may be a microorganism including an unaltered, “wild-type” GDH gene. The same comparison is applied to other genetic modifications.

An activity of the enzyme may be inactivated or decreased by deletion or disruption of a gene encoding the enzyme. The “deletion” or “disruption” of the gene refers to mutation of part or all of the gene or part or all of a regulatory sequence of the gene(e.g., a promoter or a terminator region), such that the gene is either not expressed, expressed at a reduced level, or the gene product (e.g., enzyme) is expressed with no activity or reduced activity compared to the naturally occurring gene product. The mutation may include addition, substitution, insertion, deletion, or conversion of one or more nucleotides of the gene. The deletion or disruption of a gene may be achieved by genetic manipulation such as homologous recombination, directed mutagenesis, or molecular evolution. When a cell includes a plurality of the same genes, or two or more different paralogs of a gene, one or more of the genes may be removed or disrupted. For example, inactivation or disruption of the enzyme may be caused by homologous recombination or may be performed by transforming the cell with a vector including a part of sequence of the gene, culturing the cell so that the sequence may homogonously recombine with an endogenous gene of the cell to delete or disrupt the gene, and then selecting cells, in which homologous recombination occurred, using a selection marker.

The term “gene”, as used herein, refers to a nucleic acid fragment encoding a specific protein, and the fragment may or may not include a regulatory sequence of a 5′-non coding sequence and/or 3′-non coding sequence.

A “sequence identity” of a nucleic acid or a polypeptide, as used herein, refers to the extent of identity between bases or amino acid residues of sequences obtained after the sequences are aligned so as to best match in certain comparable regions. The sequence identity is a value that is obtained by comparison of two sequences in certain comparable regions via optimal alignment of the two sequences, in which portions of the sequences in the certain comparable regions may be added or deleted compared to reference sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (e.g., the size of a range), and multiplying a result of the division by 100 to obtain the percentage of the sequence identity. The percentage of the sequence identity may be determined using a known sequence comparison program, for example, BLASTN (NCBI), CLC Main Workbench (CLC bio) and MegAlign™ (DNASTAR Inc).

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

The “genetic modification”, as used herein, includes an artificial alteration in a constitution or structure of a genetic material of a cell.

An aspect of the disclosure provides a Gluconacetobacter recombinant microorganism including a genetic modification that increases the activity of ATP-dependent protease ATPase (HslUV) particularly subunit HsIU. In some embodiments, the recombinant microorganism has enhanced (increased) cellulose productivity as compared to the same Gluconacetobacter microorganism without the genetic modification that increases the activity of ATP-dependent protease ATPase (HslUV) particularly subunit HsIU.

ATP-dependent protease ATPase (HsIUV) is a complex of heat shock proteins HsIV and HsIU, and expressed in many bacteria in response to cell stress. HsIV protein is a protease, and HsIU protein is ATPase. The complex may include a dodecameric HsIV protein and a hexameric HsIU protein. HsIV protein degrades unneeded or damaged proteins only when in complex with the hsIU protein in the ATP-bound state. The complex is thought to be the ancestor of the proteasome in eukaryotes.

HsIU may be an enzyme classified as Enzyme Code (EC) 3.4.25.2. HsIU may be from the genus Gluconacetobacter, the genus Escherichia, or the genus Haemophilus. HsIU may be derived from, for example, G. xylinus, E. coli, or Haemophilus influenzae. HsIU may be a polypeptide having a sequence identity of about 95% or more to an amino acid sequence of SEQ ID NO: 1.

In the microorganism, the genetic modification may increase expression of a gene encoding HsIU. The genetic modification may increase the copy number of the gene encoding the polypeptide having a sequence identity of about 95% or more to the amino acid sequence of SEQ ID NO: 1. The gene may have a nucleotide sequence of SEQ ID NO: 3. The genetic modification may be introduction of the gene encoding HsIU, for example, via a vehicle such as a vector. The gene encoding HsIU may exist within or outside the chromosome. The introduced gene encoding HsIU may be plural, for example, 2 or more, 5 or more, 10 or more, 50 or more, 100 or more, or 1000 or more.

The microorganism may be the genus Gluconacetobacter, for example, G. aggeris, G. asukensis, G. azotocaptans, G. diazotrophicus, G. entanii, G. europaeus, G. hansenii, G. intermedius, G. johannae, G. kakiaceti, G. kombuchae, G. liquefaciens, G. maltaceti, G. medellinensis, G. nataicola, G. oboediens, G. rhaeticus, G. sacchari, G. saccharivorans, G. sucrofermentans, G. swingsii, G. takamatsuzukensis, G. tumulicola, G. tumulisoli, or G. xylinus (also called “K. xylinu; ”).

In some embodiments, the microorganism may include a genetic modification that increases the activity of subunit HsIU of HsIUV, but does not include a genetic modification that increases the activity of subunit HsIV.

The microorganism may further include a genetic modification that decreases activity of pyrroloquinoline-quinone (PQQ)-dependent glucose dehydrogenase (GDH). The microorganism may have deletion or disruption of a gene encoding GDH. The genetic modification may have deletion or disruption of a gene encoding a polypeptide having a sequence identity of about 95% or more to an amino acid sequence of SEQ ID NO: 2. The GDH gene may have a nucleotide sequence of SEQ ID NO: 4.

Another aspect of the invention provides a method of producing cellulose, the method including culturing a Gluconacetobacter recombinant microorganism comprising a genetic modification increasing activity of ATP-dependent protease ATPase (HsIUV) subunit HsIU, in a medium to produce cellulose; and collecting the cellulose from a culture. The Gluconacetobacter recombinant microorganism is the same as described above.

The culturing may be performed in a medium containing a carbon source, for example, glucose. The medium used for culturing the microorganism may be any general medium that is suitable for host cell growth, such as a minimal or complex medium containing proper supplements. The suitable medium may be commercially available or prepared by a known preparation method. The medium used for the culturing may be a medium that may satisfy the requirements of a particular microorganism. The medium may be a medium including components selected from the group consisting of a carbon source, a nitrogen source, a salt, trace elements, and combinations thereof.

The culturing conditions may be appropriately controlled for the production of a selected product, for example, cellulose. The culturing may be performed under aerobic conditions for cell proliferation. The culturing may be performed by static culture without shaking. The culturing may be performed at a low density, in which a density of the microorganism is OD₆₀₀=0.1 or less. The density of the microorganism may be a density which gives a space not to disturb secretion of cellulose.

The term “culture conditions”, as used herein, mean conditions for culturing the microorganism. Such culture conditions may include, for example, a carbon source, a nitrogen source, or an oxygen condition utilized by the microorganism. The carbon source that may be utilized by the microorganism may include monosaccharides, disaccharides, or polysaccharides. The carbon source may include glucose, fructose, mannose, or galactose as an assimilable sugar. The nitrogen source may be an organic nitrogen compound or an inorganic nitrogen compound. The nitrogen source may be exemplified by amino acids, amides, amines, nitrates, or ammonium salts. An oxygen condition for culturing the microorganism may be an aerobic condition of a normal oxygen partial pressure, a low-oxygen condition including about 0.1% to about 10% of oxygen in the atmosphere, or an anaerobic condition including no oxygen. A metabolic pathway may be modified in accordance with a carbon source or a nitrogen source that may be actually used by a microorganism.

The method may include collecting the cellulose from the culture. The separating may be, for example, collecting of a cellulose pellicle formed on the top of the medium. The cellulose pellicle may be collected by physically stripping off the cellulose pellicle or by removing the medium. The separating may be collecting of the cellulose pellicle while maintaining its shape without damage.

Still another aspect of the invention provides a method of producing the microorganism having enhanced cellulose productivity, the method including introducing a gene encoding ATP-dependent protease ATPase (HsIUV) subunit HsIU into a Gluconacetobacter microorganism. The introducing of the gene encoding HsIU may comprise introducing a vehicle including the gene into the microorganism. In the method, the genetic modification may include amplification of the gene, manipulation of the regulatory sequence of the gene, or manipulation of the sequence of the gene itself. The manipulation may be insertion, substitution, conversion, or addition of nucleotides.

The method may further include introducing a genetic modification that decreases the activity of pyrroloquinoline-quinone (PQQ)-dependent glucose dehydrogenase (GDH) into the microorganism. The genetic modification may be deletion or disruption of the gene encoding GDH.

The Gluconacetobacter recombinant microorganism having enhanced cellulose productivity of an aspect may be used to produce cellulose in a high yield.

The method of producing cellulose of another aspect of the invention may be used to efficiently produce cellulose.

The method of producing the microorganism having enhanced cellulose productivity of still another aspect of the invention may be used to efficiently produce the microorganism having enhanced cellulose productivity.

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items.

Hereinafter, the present invention will be described in more detail with reference to Examples. However, these Examples are for illustrative purposes only, and the scope of the present invention is not intended to be limited by these Examples.

### EXAMPLE 1

### Preparation of HsIU Gene-Including G. xylinus and Production of Cellulose

In this Example, G. xylinus (Korean Culture Center of Microorganisms, KCCM 41431) and GDH gene-deleted G. xylinus were introduced with the HsIU gene, and the microorganisms introduced with the gene were cultured to produce cellulose, thereby examining effects of the gene introduction on cellulose productivity.

(1) Preparation of GDH Gene-Deleted G. xylinus

The membrane-bound pyrroloquinoline-quinone (PQQ)-dependent glucose dehydrogenase (GDH) gene in G. xylinus was inactivated by homologous recombination. A specific procedure is as follows.

To delete GDH gene by homologous recombination, fragments of the 5′- and 3′-ends of GDH gene were obtained by PCR amplification using a genomic sequence of G. xylinus as a template and a set of primers of GDH-5-F(SEQ ID NO: 5) and GHD-5-R(SEQ ID NO: 6) and a set of primers of GDH-3-F(SEQ ID NO: 7) and GHD-3-R(SEQ ID NO: 8). Further, a neo gene (nptII) fragment which is a kanamycin resistance gene derived from Tn5 was obtained by PCR amplification using a set of primers of SEQ ID NO: 12 and SEQ ID NO: 13. Three of the fragments of the 5′- and 3′-ends of GDH gene and the kanamycin resistance gene fragment were cloned into SacI and XbaI restriction sites of a pGEM-3zf vector (#P2271, Promega Corp.) using an In-fusion HD cloning kit (#PT5162-1, Clontech) to prepare pGz-dGDH. This vector was transformed into G. xylinus by electroporation. The transformed X. xylinus strain was spread on a HS-agar medium (0.5% peptone, 0.5% yeast extract, 0.27% Na₂HPO₄, 0.15% citric acid, 2% glucose, and 1.5% bacto-agar) supplemented with 100 μg/ml of kanamycin, and then cultured at 30° C. A strain having kanamycin resistance was selected to delete GDH gene. As a result, GDH gene deletion was confirmed, and this strain was designated as G. xylinus (Δgdh).

(2) Introduction of HsIU Gene

G. xylinus-derived HsIU gene (SEQ ID NO: 3) was introduced into G. xylinus and G. xylinus (Δgdh), respectively. A specific introduction procedure is as follows.

G. xylinus-derived HsIU gene was obtained by PCR using primers of SEQ ID NO: 9 and SEQ ID NO: 10 and a genomic sequence of G. xylinus as a template. This gene was introduced into SaII and PstI restriction sites of pTSa (SEQ ID NO: 11) to allow expression under Tac promoter. The strains obtained were designated as G. xylinus (pTSa-HsIU) and G. xylinus (Δgdh, pTSa-HsIU), respectively.

(3) Test of Cellulose Production

The designated G. xylinus strains were inoculated into a 250-mL flask containing 50 ml of HS medium (0.5% peptone, 0.5% yeast extract, 0.27% Na₂HPO₄, 0.15% citric acid, and 2% glucose), and cultured by static culture without shaking or under shaking at 230 rpm at 30° C. for 4 days. The products were measured.

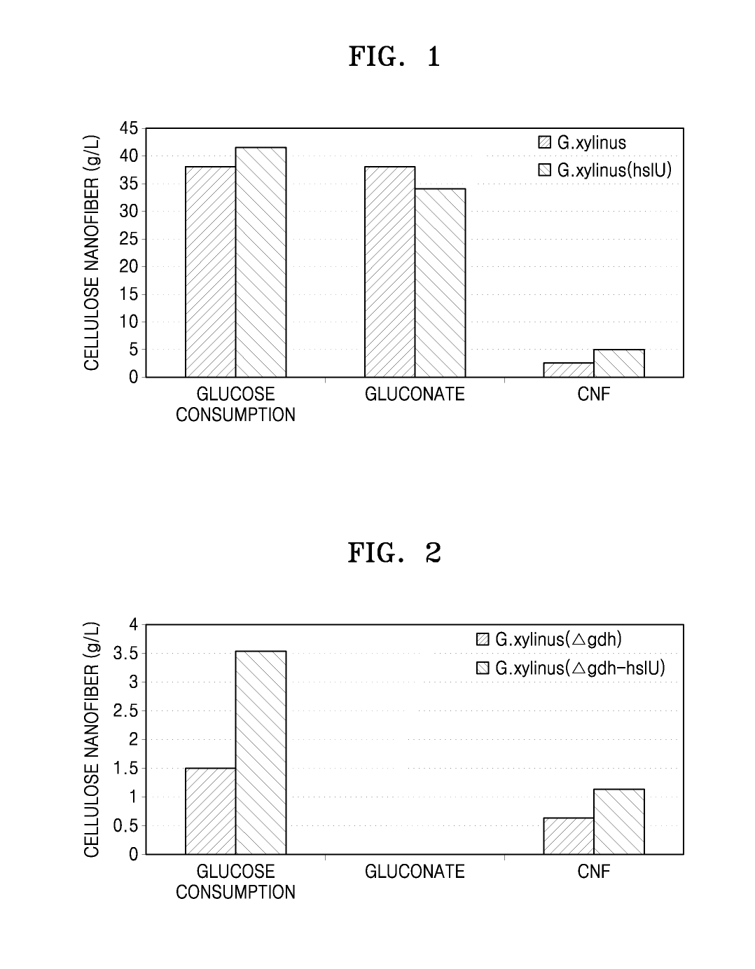

The results are given in FIGS. 1 and 2. FIG. 1 shows amounts of cellulose nanofiber (CNF) produced by G. xylinus and G. xylinus (HsIU) strains under static culture. As shown in FIG. 1, when hsIU gene was introduced into G. xylinus, the CNF production amount showed about 85% increase from 2.8 g/L to 5.2 g/L. When DPw and DPv values of the produced cellulose were measured, DPw value showed about 6% increase from 7410 to 7885, and DPv value showed about 19% increase from 4727 to 5619. Upon shaking culture, when hsIU gene was introduced into G. xylinus, the CNF production amount showed about 68% increase from 3.8 g/L to 6.4 g/L. When DPw and DPv values of the produced cellulose were measured, DPw value showed about 6% increase from 7410 to 7885, and DPv value showed about 19% increase from 4727 to 5619, indicating that hsIU introduction influenced the production amount of cellulose and properties of cellulose nanofiber. Table 1 shows physical properties of cellulose nanofiber produced by G. xylinus and G. xylinus (HsIU) strains upon static culture and shaking culture.

TABLE 1

| Culture | Strain | Mean DPw | Standard deviation (±) | DPv |
|---|---|---|---|---|
| Shaking | G. xylinus | 7410 | 288 | 4727 |
| | G. xylinus | 7885 | 21 | 5619 |
| | (HsIU) | | | |
| Static | G. xylinus | 7044 | 19 | 4029 |
| | G. xylinus | 7195 | 87 | 4290 |
| | (HsIU) | | | |

Herein, the degree of polymerization (DP) of CNF was measured as Degree of polymerization determined by viscosity measurement (DPv) and weight average degree of polymerization (DPw).

For measurement of DPw, 5 mg of freeze-dried CNF sample was derivatized at 100° C. for 48 hours by addition of 10 mL of pyridine and 1 mL of phenyl isocyanate. The derivatized CNF was added to 2 ml of methanol, and then solidified by addition of 100 mL of 70% methanol, followed by washing with water twice. Water was removed from CNF thus prepared under vacuum, and then CNF was incubated with 1 ml of tetrahydrofuran per 1 mg of CNF at 50° C. for 1 hour. Gel permeation chromatography (GPC) was used to determine a molecular weight, a molecular weight distribution, and a length distribution of CNF. A GPC test was performed on Waters Alliance e2695 separation module (Milford, Mass., USA) equipped with Waters 2414 refractive index detector and Styragel HR2, HR4, HMW7 columns. Tetrahydrofuran was used as an eluent at a flow rate of 0.5 mL/min. CNF incubated in tetrahydrofuran was filtered using a 0.15 urn syringe filter (PTFE), and then injected (injection volume: 20 uL). Polystyrene standards (PS, #140) were used to calibrate the curve.

15 mg of freeze-dried CNF was incubated in 15 mL of 0.5 M cupriethylenediamine solution for about 2 hours, and then viscosity thereof was examined using a visco pump (ACS370) and a viscometer (Ubbelohde).

FIG. 2 shows amounts of cellulose nanofiber (CNF) produced by G. xylinus (Δgdh) and G. xylinus (Δgdh-HsIU) strains under static culture. As shown in FIG. 2, when hsIU gene was introduced into G. xylinus (Δgdh), glucose consumption showed about 134% increase from 1.52 g/L to 3.56 g/L, and the cellulose production amount showed about 75% increase from 0.66 g/L to 1.16 g/L. Upon shaking culture, when hslU gene was introduced into G. xylinus (Δgdh), glucose consumption showed about 98% increase from 2.11 g/L to 4.21 g/L, and the cellulose production amount showed about 57% increase from 2.20 g/L to 3.46 g/L. Table 2 shows physical properties of cellulose nanofiber produced by G. xylinus (Δgdh) and G. xylinus (Δgdh-HsIU) strains upon static culture and shaking culture.

TABLE 2

| | | | Standard | |
|---|---|---|---|---|
| Culture | Strain | Mean DPw | deviation (±) | DPv |
| Shaking | G. xylinus | 7020 | 45 | 4060 |
| | (Δgdh) | | | |
| | G. xylinus | 8008 | 75 | 4412 |
| | (HsIU-Δgdh) | | | |
| Static | G. xylinus | 6793 | 119 | 3967 |
| | (Δgdh) | | | |
| | G. xylinus | 8451 | 25 | 3866 |
| | (Δgdh-HsIU) | | | |

As described above, when HsIU gene is introduced into G. xylinus, and optionally, Δgdh is also introduced thereto, cellulose productivity was remarkably increased, and physical properties of the produced cellulose nanofiber were also remarkably increased.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant Gluconacetobacter microorganism comprising a genetic modification that increases the activity of ATP-dependent protease ATPase (HsIUV) subunit HsIU.

2. The microorganism of claim 1, wherein the genetic modification increases the expression of a gene encoding HsIU.

3. The microorganism of claim 1, wherein HsIU is an enzyme classified as Enzyme Code (EC) 3.4.25.2.

4. The microorganism of claim 2, wherein the microorganism comprises a gene encoding HsIU from G. xylinus, E. coli, or Haemophilus influenzae.

5. The microorganism of claim 2, wherein HsIU is a polypeptide having a sequence identity of 95% or more to an amino acid sequence of SEQ ID NO: 1.

6. The microorganism of claim 1, wherein the genetic modification is an increase in the copy number of a gene encoding a polypeptide having a sequence identity of 95% or more to an amino acid sequence of SEQ ID NO: 1.

7. The microorganism of claim 2, wherein the gene has a nucleotide sequence of SEQ ID NO: 3.

8. The microorganism of claim 1, wherein the microorganism is Gluconacetobacter xylinus.

9. The microorganism of claim 1, further comprising a genetic modification that decreases activity of pyrroloquinoline-quinone (PQQ)-dependent glucose dehydrogenase (GDH).

10. The microorganism of claim 1, wherein a gene encoding GDH is deleted or disrupted in the microorganism.

11. The microorganism of claim 9, wherein the genetic modification that decreases activity of pyrroloquinoline-quinone (PQQ)-dependent glucose dehydrogenase (GDH) comprises a deletion or disruption of a gene encoding a polypeptide having a sequence identity of 95% or more to an amino acid sequence of SEQ ID NO: 2.

12. A method of producing cellulose, the method comprising:
culturing a Gluconacetobacter recombinant microorganism of claim 1; and
collecting the cellulose from a culture.

13. The method of claim 12, wherein the genetic modification increases the expression of a gene encoding HsIU.

14. The method of claim 12, wherein HsIU is an enzyme classified as Enzyme Code (EC) 3.4.25.2.

15. The method of claim 12, wherein the microorganism is Gluconacetobacter xylinus.

16. The method of claim 12, wherein HsIU is a polypeptide having a sequence identity of 95% or more to an amino acid sequence of SEQ ID NO: 1.

17. The method of claim 12, wherein the genetic modification increases the copy number of a gene encoding an HsIU polypeptide having a sequence identity of 95% or more to an amino acid sequence of SEQ ID NO: 1.

18. The method of claim 12, wherein the microorganism further comprises a genetic modification that decreases the activity of PQQ-dependent glucose dehydrogenase (GDH).

19. A method of producing a microorganism having enhanced cellulose productivity, the method comprising introducing a gene encoding ATP-dependent protease ATPase (HsIUV) subunit HsIU into a Gluconacetobacter microorganism.

20. The method of claim 19, further comprising introducing a genetic modification that decreases the activity of PQQ-dependent glucose dehydrogenase (GDH).


SEQUENCE LISTING (13 sequences)

SEQ ID NO: 1 - protein, 437 aa, Komagataeibacter xylinus
MEIPNHTPREIVSELDRFIIGQGDAKRAVAIALRNRWRRAQLPDALREEVVPKNILMIGPTGCGKTEIARRLAKLAQAPFLKVEATKFTEVGYVGRDVESIIRDLIEVSINMLRDLRRRDVEANAGDAAEKLLLDALVGEGASAETRNKFRRMLRAGELEHKEVEISIAEGGNPAQSDMPNMTPGTVINFSDMMKGFMNRVPQQKRMTVAAARAALIRQEADRMLDTEALTREAVAHAQDHGIVFLDEIDKVCARAAEGGARGGDVSREGVQRDLLPLIEGTTVSTKYGPVRTDHILFIASGAFHIAKPSDLLPELQGRLPIRVELASLTREDLRRILTEPEHSLLKQYVALLGTEDVKLSFSDGAIDALAELAADINERVENIGARRLATVLERLLEDVSFTAADRKGEAVLIEAADVQARVAPLARKGDLSRFIL

SEQ ID NO: 2 - protein, 796 aa, Komagataeibacter xylinus
MNSLMRSAPLLAAAIAVCALTGLYLLGGGLWLCLIGGSFYYVVAGVLLLVTAVLLARRQAMALTVYAVLLLGTMVWAVQEAGFDFWALAPRGDILVPIGIVLALPWVTRHLQPASPATHLPLFGAIGAAVVVVGAALTQDPQDIAGSLPPVAQNAPEPGDAHQMPDEDWQAYGRTQFGDRFSPLKQVNASNVGKLKVAWTFRTGDLRGPNDPGEITDEVTPIKIRDTLYLCTPHQILFALDAKTGQQRWKFDPKLAYNPTFQHLTCRGVSYHEDRADDAQAADGAAAPAECARRIFLPTNDGQLFALDAATGARCASFGNNGVVNLQDGMPVKTLGFYEPTSPPVVTDTTVIVSGAVTDNYSTHEPSGVTRGFDVHTGALKWAFDPGNPDPNEMPSEHHTFVPNSPNSWITSSYDAKLDLIYIPMGVQTPDIWGGNRGADAERYASSIVALNATTGRLVWSYQTVHHDLWDMDIPAQPSLVDIRNEQGEVIPTLYAPAKTGNIFVLDRRNGQPVVPAPEHPVPQGAAPGDHVSPTQPFSELSFRPKKLLTDADMWGGTMYDQLVCRIMFHRLRYEGTFTPPSLQGTLVFPGNLGMFEWGGLAVDPVRQIAIANPIAIPFVSKLIPRGPNNPATPDKSLPSGSESGVQPQFGVPYGVDLHPFLSPFGLPCKQPAWGYMSGIDLRTNKIVWKHRNGTIRDSAPLPLPIKMGVPSLGGPLTTAGGVAFLTSTLDYYIRAYDVTNGQVLWQDRLPAGGQSTPMTYAVDGKQYIVTADGGHGSFGTKLGDYIVAYSLPDGN

SEQ ID NO: 3 - DNA, 1314 bp, Komagataeibacter xylinus
atggaaatacccaatcataccccgcgtgagatcgtctccgaactcgaccgtttcatcatcgggcagggtgatgccaagcgggccgtggccattgcgctgcgcaaccgctggcgccgcgcccagttgcccgatgcgctgcgtgaggaggtggtgcccaagaacatcctgatgatcgggcccaccggctgcggcaagaccgagatcgcccgccgcctcgccaagctcgcgcaggcgccgttcctcaaggtcgaggccaccaaattcaccgaagtgggttacgtgggccgcgatgtggaaagcatcatccgcgacctgatcgaggtctcgatcaacatgctgcgcgacctgcggcgcagggatgtcgaggccaatgccggggacgccgccgagaagctcctgctcgatgcgctggtgggcgagggggcctcggccgagacccgcaacaagttccgccgcatgctgcgcgcgggcgagcttgagcacaaggaagtcgagatatcgattgccgagggcggcaacccggcccagtccgacatgccgaacatgacgccgggcaccgtcatcaacttctcggacatgatgaagggcttcatgaaccgcgtgccgcagcaaaagcgcatgacagtggccgcggcccgtgccgccctgatccggcaggaagcggaccggatgctcgataccgaggccctgacgcgcgaggccgtggcccacgcgcaggaccacggcatcgtctttctcgacgagatcgacaaggtgtgcgcccgtgccgccgaagggggcgcgcggggcggcgatgtctcgcgtgagggcgtgcagcgcgacctgctgccgctgatcgagggcaccaccgtctcgaccaaatacggcccggtgcgcacggatcatatcctgttcattgcatccggcgcgttccatattgccaagccatccgacctgttgcccgagttgcaggggcggttgcccatccgggtggaactggcctcgcttacgcgcgaggacctgcgtcgcatcctgaccgagccggagcattcactgctcaagcagtatgttgccctgctgggcacggaggatgtgaaactgtcgttcagcgacggggcgatagacgcgctggcggagcttgcggcggatattaacgagcgggttgaaaatatcggcgcgcggcgtctggccaccgtgcttgagcgccttctggaggatgtgtcctttaccgcagccgaccgcaagggcgaggctgtgctgatcgaggctgccgacgtgcaggccagggtcgcacccctggcccgcaagggtgatctgagccgctttatcctgtag

SEQ ID NO: 4 - DNA, 2391 bp, Komagataeibacter xylinus
atgaatagcctcatgcgctcggctccccttctcgctgcggccattgccgtctgcgccctgacgggtctctacctgctgggaggcgggctatggctgtgtctcatcggcggctccttttattatgttgtcgccggtgtgctgctgctggtcacggccgtgctgctggcgcggcggcaggccatggcgcttacggtctatgccgtgctcctgctcggcacgatggtgtgggccgtgcaggaagccgggtttgatttctgggcgctcgcaccgcggggcgatattctggtgcccatcggcatcgtgctcgccctgccgtgggtcacacgtcacctgcagcctgccagccccgccacccacctgcccctgttcggcgcaattggcgccgccgtggtcgtcgttggcgcggccctgacgcaggacccgcaggatatcgcgggcagcctgcccccagtcgcgcagaatgcccccgagccgggcgatgcccaccagatgcctgatgaggactggcaggcctatggccgcacccagttcggtgaccggttctccccgctcaagcaggtcaatgccagtaatgtcggcaaactgaaggtggcctggaccttccgcaccggcgacctgcgcggccccaatgaccccggtgaaatcaccgatgaggtcacccccatcaagatccgtgatacgctctatctgtgcaccccccaccagatcctgttcgcgctcgatgcgaagaccggccagcagcggtggaagtttgaccccaagctggcctacaaccccaccttccagcacctgacctgccgtggcgtgtcctatcatgaggacagggcggatgacgcgcaggcagccgatggtgccgcagccccggccgagtgcgcgcgccgcatcttcctgcccaccaatgatggccagcttttcgcgctcgatgccgcaaccggcgcgcgctgcgcaagctttggcaataatggcgtggtgaacctgcaggacggcatgccggtcaagacgctgggcttttatgaaccgacctcccccccggtcgtgaccgataccaccgtgatcgtgtccggcgccgtgaccgacaactattccacgcatgagccttcgggggttacgcgcggcttcgacgtgcataccggcgcgctgaaatgggcgttcgaccccggcaatcccgatccgaacgagatgccgtccgagcaccacaccttcgtgccgaactcacccaattcgtggatcacgtcgtcctatgatgccaagctggacctgatctacatccccatgggcgtgcagacgcccgatatctggggcggcaaccgcggcgccgatgccgagcgctatgcaagctccatcgtggcgctgaacgccaccaccggcaggctggtctggtcctaccagaccgtgcaccacgacctgtgggacatggacatccccgcccagcccagcctggtcgatatccgcaacgaacagggcgaggtcatccccaccctgtatgccccggccaagaccggcaacatcttcgtgcttgaccggcgcaacggccagcccgtggtgcccgcccccgagcacccggtgccgcagggcgcagcccctggcgatcacgtttcgcccacgcagcctttctcggagctgagcttccgccccaagaagctgctgaccgatgccgatatgtggggcggcacgatgtatgaccagctggtctgccgcatcatgttccaccgcctgcgctacgaaggcacattcacgccgccttcgctgcagggcacgctggtcttccccggcaatctcggcatgttcgaatggggcggccttgcggtcgaccccgtgcgccagatcgcgattgccaaccccatcgccattccgttcgtctccaaactgatcccgcgcggcccgaacaacccggcaacgcctgacaagtccctgccctcgggctcggagagtggcgtgcagccgcagtttggcgtgccttacggcgtggacctgcatccgttcctctcgccgtttggcctgccgtgcaagcagcccgcctggggctacatgtcgggcatcgacctgcgcaccaacaagatcgtgtggaagcaccgcaacggcacgatccgtgacagcgcaccgctgcccctgcccatcaagatgggcgtgcccagccttggcggcccgctcaccacggcgggtggcgtggccttcctcacttccacgctcgattactacatccgcgcctatgacgtgacgaacggccaggtgctgtggcaggaccgcctgcctgccggtggccagtccacgcccatgacctatgcggtcgatggcaagcagtacatcgtcacggccgatggcggccacgggtcgttcggcaccaaactcggcgactacatcgtcgcctacagcctgcctgacgggaactga

SEQ ID NO: 5 - DNA, 35 bp, Artificial Sequence
  Synthetic gdh 5 prime terminal forward primer
tagaatactcaagcttggagctaccagaccgtcca

SEQ ID NO: 6 - DNA, 33 bp, Artificial Sequence
  Synthetic gdh 5 prime terminal reverse primer
tcagaccccgtagaacaaacatgccaaggttgc

SEQ ID NO: 7 - DNA, 33 bp, Artificial Sequence
  Synthetic gdh 3 prime terminal forward primer
caacaccttcttcacttgaatggggtggccttg

SEQ ID NO: 8 - DNA, 35 bp, Artificial Sequence
  Synthetic gdh 3 prime terminal reverse primer
tatagggcgaattcgggcaggcggtcctgccacag

SEQ ID NO: 9 - DNA, 43 bp, Artificial Sequence
  Synthetic hslU forward primer
gggatcctctagagtcgacatggaaatacccaatcataccccg

SEQ ID NO: 10 - DNA, 46 bp, Artificial Sequence
  Synthetic hslU reverse primer
ccaagcttgcatgcctgcagctacaggataaagcggctcagatcac

SEQ ID NO: 11 - DNA, 3128 bp, Artificial Sequence
  Synthetic pCSa vector
gaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaagtcggacgcgatgcgtccggcgtagaggatccggagcttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacagggacgagctattgattgggtaccgagctcgaattcgtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaaggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacatagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttttttaaggcagttattggtgcccttaaacgcctggttgctacgcctgaataagtgataataagcggatgaatggcagaaattc

SEQ ID NO: 12 - DNA, 34 bp, Artificial Sequence
  Synthetic Km-F primer
tcacgccgccttcgcgtgaagaaggtgttgctga

SEQ ID NO: 13 - DNA, 33 bp, Artificial Sequence
  Synthetic Km-R primer
aacaccagcgtgcccttctacggggtctgacgc